(19) United States
(12) Patent Application Publication
(10) Pub. No.: US 2011/0272352 A1
(43) Pub. Date: Nov. 10, 2011

(54) MEMBRANE ELECTROLYZER AND HEMODIALYSIS SYSTEM USING THE SAME

(75) Inventor: James Braig, Piedmont, CA (US)

(73) Assignee: C-TECH BIOMEDICAL, INC., Anaheim, CA (US)

(21) Appl. No.: 13/101,808

(22) Filed: May 5, 2011

Related U.S. Application Data
(60) Provisional application No. 61/331,502, filed on May 5, 2010.

(51) Int. Cl. B01D 61/26; C12M 1/12; C25B 1/00; B01D 61/28

(52) U.S. Cl. 210/632; 210/192

(57) ABSTRACT

A sorbent hemodialysis system includes a dialyzer configured to receive a flow of clean dialysate from a reservoir and to output an unclean dialysate flow. The system also includes a sorbent component having a urease section and a sorbent section through which the unclean dialysate flow from the dialyzer passes, wherein the sorbent component removes urea from the dialysate. The system further comprises a membrane electrolyzer that receives at least a portion of said clean dialysate flow and separates the dialysate flow into an acidic component flow and a base component flow. A mixing conduit combines the base component flow from the membrane electrolyzer and an output dialysate solution from the urease section of the sorbent component to separate the dialysate solution into an ammonia gas amount and ammonia liquid amount. A gas vent is used to vent the ammonia gas amount, and the sorbent section with a suitable amount of zirconium phosphate (ZrP) removes the ammonia liquid amount from the unclean dialysate flow before flowing the clean dialysate to the reservoir. The system can further include a second mixing conduit upstream of the sorbent section of the sorbent component, the second mixing conduit combining the acidic component flow and the ammonia liquid amount in the dialysate solution to increase the pH of the dialysate solution to about 7.5 prior to returning to the reservoir.

### CROSS-REFERENCE TO RELATED APPLICATIONS

This application claims the benefit of U.S. Provisional Application No. 61/331,502, filed May 5, 2010, the entire contents of which are hereby incorporated by reference and should be considered a part of this specification.

### BACKGROUND

1. Field

The present invention is directed to a sorbent hemodialysis system, and more particularly to a sorbent hemodialysis system with a membrane electrolyzer.

2. Description of the Related Art

In conventional sorbent based hemodialysis systems, urease enzyme is used to convert urea to NH₄+ which is then removed from the dialysate via ion exchange with ZrP (NaHZrP) in a sorbent cartridge. A typical sorbent cartridge designed for every other day dialysis treatments removes about 30 gm of urea and contains about 1,767 grams of ZrP.

The typical sorbent based hemodialysis process 100 is shown in FIG. 1. A dialysate D, which is a “normal” saline solution having a pH of approximately 7.5, is pumped via pump P from a reservoir R to a dialyzer 10 (e.g., artificial kidney), which has an urea supply pump 12 and mixer 14, where it is loaded with urea. The dialysate D loaded with urea is then pumped though the sorbent cartridge 1 that contains urease and sorbents to remove the urea, after which the clean dialysate is returned to the reservoir R.

However, conventional sorbent dialysis treatment can be costly, particularly for patients that must receive treatment every day or every other day. One contributor to the cost of sorbent based dialysis is the cost of the sorbent cartridge, which costs approximately $30 per cartridge, of which about $15 is the cost of the ZrP in the sorbent cartridge (e.g., about 1,767 grams of ZrP as noted above), based on production volumes. Therefore removing the Ammonia in a conventional sorbent hemodialysis system via the ZrP is expensive, as ZrP represents about 50% of the total cost of a standard sorbent dialysis cartridge

A need exists for an improved and less costly sorbent cartridge and dialysis system.

### SUMMARY

In accordance with one embodiment, a sorbent hemodialysis system is provided. The system comprises a dialyzer configured to receive a flow of clean dialysate from a reservoir, the dialyzer configured to output an unclean dialysate flow. The system also comprises a sorbent component having a urease section and a sorbent section through which the unclean dialysate flow from the dialyzer passes, the sorbent component configured to remove urea from the unclean dialysate flow. The system further comprises a membrane electrolyzer configured to receive at least a portion of said clean dialysate flow and to separate the dialysate flow into an acidic component flow and a base component flow. The system also comprises a mixing conduit configured to combine the base component flow from the membrane electrolyzer and an output dialysate solution from the urease section to separate the dialysate solution into an ammonia gas amount and ammonia liquid amount. A gas vent is configured to vent the ammonia gas amount, and the sorbent section is configured to have an amount of zirconium phosphate (ZrP) suitable to remove the ammonia liquid amount from the unclean dialysate flow before flowing the clean dialysate to the reservoir. In some embodiments, the system further comprises a second mixing conduit upstream of the sorbent section, the second mixing conduit configured to combine the acidic component flow and the ammonia liquid amount in the dialysate solution to increase the pH of the dialysate solution to about 7.5 prior to returning the clean dialysate flow to the reservoir.

In accordance with another embodiment, a method for operating a dialysate flow circuit of a sorbent hemodialysis system is provided. The method comprises pumping a clean dialysate flow from a reservoir through a dialyzer, the dialyzer configured to output an unclean dialysate flow, flowing the unclean dialysate flow through a sorbent component having a urease section and a sorbent section, and flowing at least a portion of the clean dialysate flow through a membrane electrolyzer to separate the portion of the clean dialysate flow into an acidic component flow and a base component flow. The method further comprises combining the base component flow with a dialysate solution output from the urease section to thereby separate an ammonia amount in the dialysate solution into an ammonia gas amount and ammonia liquid amount, venting the ammonia gas amount, combining the acidic component flow with the dialysate solution having the ammonia liquid amount at a location upstream of the sorbent section, and removing the ammonia liquid amount from the dialysate solution via the sorbent section.

### BRIEF DESCRIPTION OF THE DRAWINGS

FIG. 1 is a schematic diagram of a conventional sorbent dialysis system

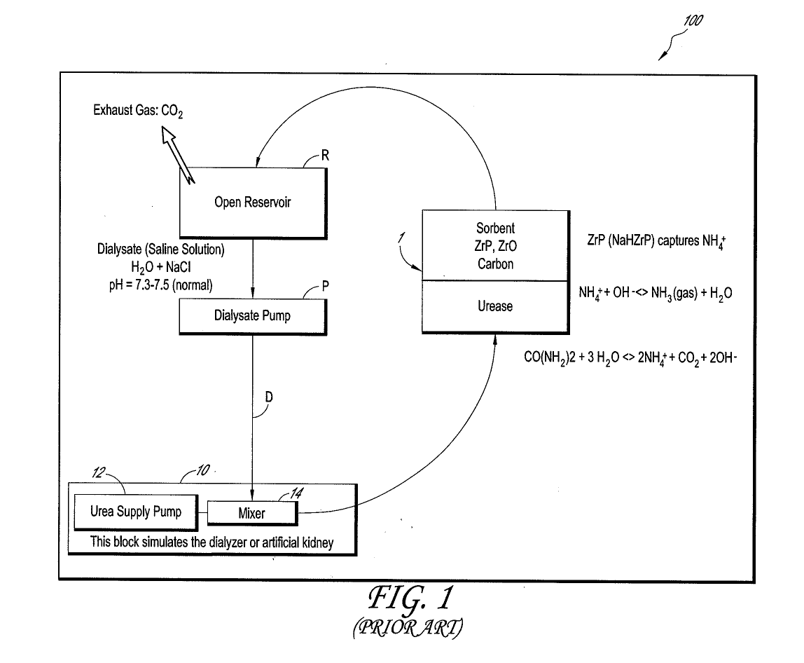

FIG. 2 is a schematic diagram of a portion of one embodiment of a sorbent dialysis system having a sorbent cartridge with a membrane electrolyzer.

FIG. 3 is a table of the balance between NH₄ liquid and NH₃ gas at various pH levels.

FIG. 4 is a schematic diagram of a membrane electrolyzer.

### DETAILED DESCRIPTION

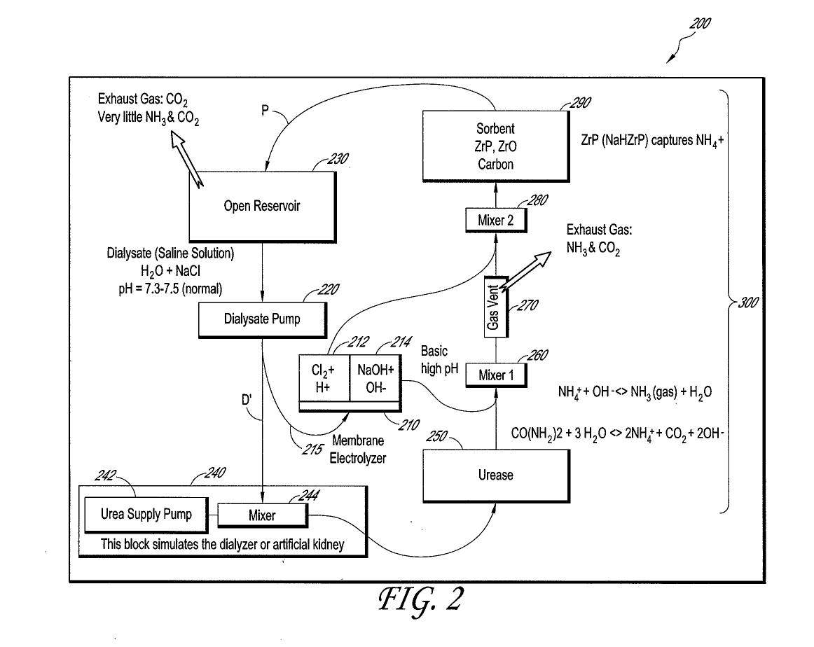

FIG. 2 shows a portion of one embodiment of an improved sorbent hemodialysis system 200. In particular, FIG. 2 shows a dialysate flow path or circuit P of the hemodialysis system 200. In the illustrated embodiment, a membrane electrolyzer 210 receives at least a portion 215 of a dialysate D′ flow pumped by a dialysate pump 220 in fluid communication with a dialysate reservoir 230. The remaining dialysate flow D′ is pumped through the dialyzer 240, which can have a urea supply pump 242 and a mixer 244. The dialysate flow loaded with urea D exits the dialyzer 240 and passes through a urease section 250.

The membrane electrolyzer 210 splits the dialysate flow 215 into an acidic component 212 and a base component 214. The base component 214 is added to the dialysate flow D downstream of the urease section 250 via a mixer 260, and is used to raise the pH of the dialysate flow D to effect “blowing off” of ammonia and carbon dioxide as a gas via a gas vent 270. Then, the acidic component 212 is recombined with the dialysate flow D via a mixer 280 to assure the overall pH of the dialysate flow D is unaffected (e.g., the pH of the dialysate flow D is returned to it's normal pH of 7.5). The dialysate flow D passes from the mixer 280 through a sorbent section 290, which can contain an appropriate amount of ZrP, before the clean dialysate D′ is returned to the reservoir 230. As shown in FIG. 2, the reservoir 230 can be an open reservoir and can exhaust gas in the form of NH₃ and CO₂. Advantageously, using the membrane electrolyzer 210 allows for the recombination of the output streams of the acidic and base components 212, 214 and insures the pH of the dialysate D returns to the pre-electrolyzer 210 level without requiring any precision in mixing the acidic and base components 212, 214 with the dialysate flow D.

With continued reference to FIG. 2, the sorbent component 300 is split into two components, the urease section 250 and the sorbent section 290. In one embodiment, sorbent component 300 can be a single cartridge that includes the urease section 250, sorbent section 290, mixers 260, 280 and gas vent 270. In another embodiment, the urease section 250, sorbent section 290, mixers 260, 280 and gas vents 270 can be separate components. The split in the sorbent component 300 into the urease section 250 and sorbent section 290 advantageously allows access to the ammonia (NH₃) gas via the urease section 250 and mixer 260. However, because the urease is not consumed, the urease section 250 can advantageously be used for more than one treatment.

With continued reference to FIG. 2, the portion 215 of the dialysate flow D′ that is diverted to the membrane electrolyzer 210, which can be a reusable component, generates two fluid flow paths. The high pH fluid is mixed with the output of the urease, via mixer 260, to increase the pH of the dialysate loaded with urea D so that the equilibrium favors the NH₃ gaseous phase. Following this mixing, the NH₃ and CO2 are degassed from the solution (e.g., via the gas vent 270).

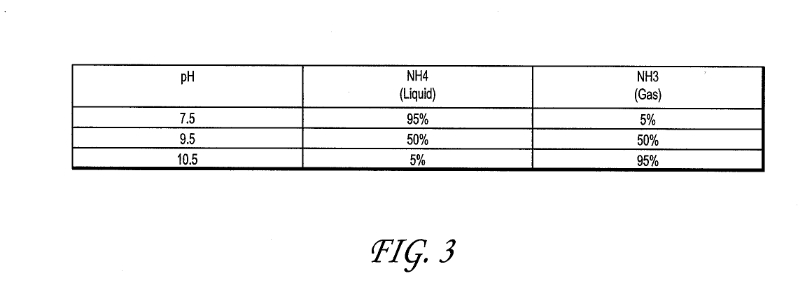

As shown in the table in FIG. 3, the equilibrium between liquid NH₄+ and gas NH₃ is dependent on pH. At the normal dialysate D solution pH of 7.5, 95% of the ammonia is in the form of liquid and is adsorbent by ZrP in a sorbent cartridge. Assuming the pH of the solution can be pushed up to 10.5, approximately 95% of the ammonia will be removed in this stage (e.g., via the gas vent 270). Following degasification the Acidic stream 212 from the membrane electrolyzer 210 is mixed back in with the solution, via mixer 280, and the net effect of the membrane electrolyzer 210 on the pH of the solution is negated. Advantageously, the pH of the dialysate solution returns to normal and the dialysate flows onto the sorbent section 290 in the remainder of the sorbent cartridge or component 300. As a result of the degasification of ammonia (NH₃) via the gas vent 270, only about 5% as much NH₄+ will need to be removed by the sorbent section 290 when the pH of the solution has been adjusted up to 10.5. This advantageously reduces the ZrP load required in the sorbent section 290 by 95%, which can reduce the cost of the sorbent cartridge or component 290 by about half (e.g., reduce the cost by about $14.25 based on the estimated costs noted above). In other embodiments, where the pH of the dialysate solution is adjusted to levels lower than 10.5, lower amounts of ammonia gas will be generated and can be vented via the gas vent 270, which will result in proportionately lower cost reductions. Any pH above approximately 9.3 (the pKa of the dialysate solution), will advantageously make a dramatic improvement in the amount of NH₄+ that needs to be adsorbed by the ZrP in the sorbent section 290. Accordingly, raising the pH of the dialysate solution D advantageously allows shifting of the Ammonia equilibrium to gas, which can then be removed by “blowing it off” rather than via adsorption into the ZrP of the sorbent section 290 of the sorbent cartridge 300.

In the sorbent hemodialysis system 200 in FIG. 2, the dialysate solution flow D that flows through the sorbent section 290 will be higher than the clean dialysate D′ flow that flows into the dialyzer 24. That is, the dialyzer 240 is operated in a “semi” bypass mode, which may provide for increased absorbance of some toxins in the sorbent section 290 of the sorbent cartridge 300 as the absorbers will effectively get a “second chance” at absorbing a portion of the dialysate flow stream.

The gas that is vented via the gas vent 270, if left untreated, may present an odor. In one embodiment, the sorbent hemodialysis system 200 can vent the gas directly outdoors to minimize the odor perceived by the user. In another embodiment, the vented ammonia gas can be captured in a lower cost sorbent (e.g., kitty litter). In still another embodiment, the vented ammonia gas can be bubbled through an acidic water mixture to convert it into a NH₄+ solution, which can then be disposed after the dialysis treatment.

With continued reference to FIG. 2, the sorbent hemodialysis system 200 can be operated so that the membrane electrolyzer 210 separates the portion of the dialysate flow 215 into the acidic component 212 and base component 214 without affecting or interfering with the flow of blood through the dialyzer 240. In one embodiment, the sorbent hemodialysis system 200 can be operated so that the membrane electrolyzer 210 separates the portion of the dialysate flow 215 into the acidic component 212 and base component 214, while the system 200 is not connected to a patient.

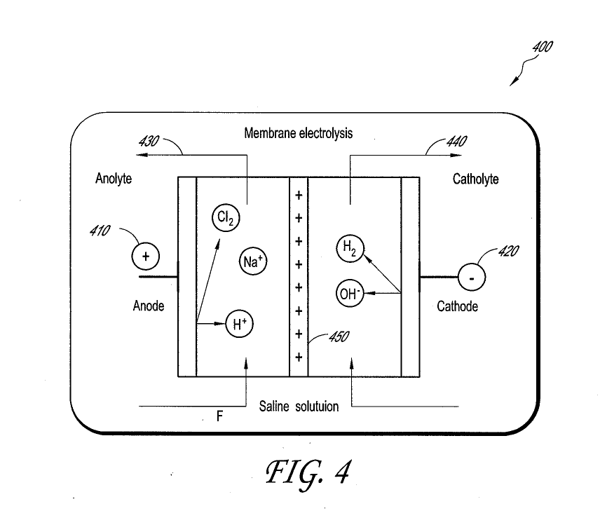

FIG. 4 shows one embodiment of a membrane electrolyzer 400. The membrane electrolyzer 400 has an anode 410 and a cathode 420. The electrolyzer 400 receives an input flow F, which in the illustrated embodiment is a saline solution, and produces an anolyte 430 and catholyte 440. A membrane 450 separates the anode loop or anolyte 430 from the cathode loop or catholyte 440. The membrane electrolyzer 400 is operated to produce a cathodic reduction reaction and an anodic oxidation reaction, which result in the separation of the anolyte 430 and catholyte 440. In the system of FIG. 2, such reactions result in the separation of the ammonia gas (NH₃) and ammonia liquid (NH₄).

Of course, the foregoing description is that of certain features, aspects and advantages of the present invention, to which various changes and modifications can be made without departing from the spirit and scope of the present invention. Moreover, the sorbent hemodialysis system need not feature all of the objects, advantages, features and aspects discussed above. Thus, for example, those skill in the art will recognize that the invention can be embodied or carried out in a manner that achieves or optimizes one advantage or a group of advantages as taught herein without necessarily achieving other objects or advantages as may be taught or suggested herein. In addition, while a number of variations of the invention have been shown and described in detail, other modifications and methods of use, which are within the scope of this invention, will be readily apparent to those of skill in the art based upon this disclosure. It is contemplated that various combinations or subcombinations of these specific features and aspects of embodiments may be made and still fall within the scope of the invention. Accordingly, it should be understood that various features and aspects of the disclosed embodiments can be combined with or substituted for one another in order to form varying modes of the discussed sorbent hemodialysis system.

What is claimed is:

1. A sorbent hemodialysis system, comprising:
a dialyzer configured to receive a flow of clean dialysate from a reservoir, the dialyzer configured to output an unclean dialysate flow;
a sorbent component having a urease section and a sorbent section through which the unclean dialysate flow from the dialyzer passes, the sorbent component configured to remove urea from the dialysate;
a membrane electrolyzer configured to receive at least a portion of said clean dialysate flow and to separate the clean dialysate flow into an acidic component flow and a base component flow;
a mixing conduit configured to combine the base component flow from the membrane electrolyzer and an output dialysate solution from the urease section to thereby separate the dialysate solution into an ammonia gas amount and ammonia liquid amount; and
a gas vent configured to vent the ammonia gas amount, the sorbent section configured to have an amount of zirconium phosphate (ZrP) suitable to remove the ammonia liquid amount from the unclean dialysate flow before flowing the clean dialysate to the reservoir.

2. The system of claim 1, wherein the base component flow is in an amount such that the ammonia gas amount of the dialysate solution is 95% of the solution and the ammonia liquid amount of the dialysate solution is 5% of the solution.

3. The system of claim 1, further comprising a second mixing conduit upstream of the sorbent section, the second mixing conduit configured to combine the acidic component flow and the ammonia liquid amount in the dialysate solution to increase the pH of the dialysate solution to about 7.5 before returning the dialysate to the reservoir.

4. The system of claim 1, wherein the sorbent component is a sorbent cartridge, the sorbent section having an amount of zirconium phosphate that is lower than in conventional sorbent cartridges.

5. The system of claim 1, wherein the amount of zirconium phosphate in the sorbent cartridge is approximately 95% lower than in conventional sorbent cartridges.

6. A method for operating a dialysate flow circuit of a sorbent hemodialysis system, comprising:
pumping a clean dialysate flow from a reservoir through a dialyzer, the dialyzer configured to output an unclean dialysate flow;
flowing the unclean dialysate flow through a sorbent component having a urease section and a sorbent section;
flowing at least a portion of the clean dialysate flow through a membrane electrolyzer to separate the portion of the clean dialysate flow into an acidic component flow and a base component flow;
combining the base component flow with a dialysate solution output from the urease section to thereby separate an ammonia amount in the dialysate solution into an ammonia gas amount and ammonia liquid amount;
venting the ammonia gas amount;
combining the acidic component flow with the dialysate solution having the ammonia liquid amount at a location upstream of the sorbent section; and
removing the ammonia liquid amount from the dialysate solution via the sorbent section.

7. The method of claim 6, wherein the base component flow is in an amount such that the ammonia gas amount of the dialysate solution is 95% of the solution and the ammonia liquid amount of the dialysate solution is 5% of the solution.

8. The method of claim 6, wherein the sorbent component is a sorbent cartridge, the sorbent section having an amount of zirconium phosphate that is lower than in conventional sorbent cartridges.

9. The method of claim 6, wherein the amount of zirconium phosphate in the sorbent cartridge is approximately 95% lower than in conventional sorbent cartridges.
